(19) United States
(12) Patent Application Publication
(10) Pub. No.: US 2014/0150785 A1
(43) Pub. Date: Jun. 5, 2014

(54) ELECTRONIC CIGARETTE OR INHALER

(71) Applicant: Vire, L.L.C., Grosse Pointe, MI (US)

(72) Inventors: Michael J. Malik, Birmingham, MI (US); Brooke Malik, Birmingham, MI (US); Elena Elder, Orchard Lake, MI (US); Alan Dolezal, Ronan, MT (US)

(73) Assignee: Vire, L.L.C., Grosse Pointe, MI (US)

(21) Appl. No.: 14/097,627

(22) Filed: Dec. 5, 2013

Related U.S. Application Data
(60) Provisional application No. 61/733,522, filed on Dec. 5, 2012.
(60) Provisional application No. 61/733,528, filed on Dec. 5, 2012.
(60) Provisional application No. 61/882,767, filed on Sep. 26, 2013.

(51) Int. Cl. A61M 15/06; A61M 16/10; A24F 47/00

(52) U.S. Cl. CPC A61M 15/06; A24F 47/008; A61M 16/1075
USPC 128/202.21; 128/203.27

(57) ABSTRACT

An electronic inhaler includes a cylindrical body, a liquid container inside the cylindrical body that contains a liquid including an additive, and a heating element to heat the liquid including the additive to a vapor. An air passage is located between the cylindrical body and the liquid container and is in communication with an air outlet to allow the vapor to flow through the air outlet.

### REFERENCE TO RELATED APPLICATION

This application claims priority to U.S. Provisional Patent Application Nos. 61/733,522 filed Dec. 5, 2012, 61/733,528 filed Dec. 5, 2012 and 61/882,767 filed Sep. 26, 2013.

### BACKGROUND OF THE INVENTION

An electronic cigarette or inhaler can be used as a substitute for a tobacco cigarette. When a person provides suction at one end of the electronic cigarette, nicotine can be inhaled into the person's lungs without the inhalation of smoke. An aerosol mist can be exhaled to simulate the smoke created by an actual tobacco cigarette.

### SUMMARY OF THE INVENTION

An electronic inhaler includes a cylindrical body, a liquid container inside the cylindrical body that contains a liquid including an additive, and a heating element to heat the liquid to a vapor. An air passage is located between the cylindrical body and the liquid container and is in communication with an air outlet to allow the vapor to flow through the air outlet.

An electronic inhaler includes an atomization nozzle including a cylindrical body, a liquid container inside the cylindrical body that contains a liquid including an additive, a heating element to heat the liquid to a vapor, and an air passage located between the cylindrical body and the liquid container in communication with an air outlet to allow the vapor to flow through the air outlet. The additive is one of a pharmaceutical, a vitamin, a flavor, a dietary supplement, and an herb. The electronic inhaler also includes a power source electrically and mechanically connected to the atomization nozzle.

Other features and advantages of the present invention will become apparent from the following description of the invention, which refers to the accompanying drawings.

### BRIEF DESCRIPTION OF THE DRAWINGS

FIG. 1 illustrates a perspective view of an electronic atomization inhaler;

FIG. 2 illustrates a perspective exploded view of an atomization nozzle of the electronic atomization inhaler;

FIG. 3 is a sectional view along line A-A of FIG. 2;

FIG. 4 is a cross-sectional view of the atomization nozzle of the electronic atomization inhaler;

FIG. 5 is a cross-sectional view of the atomization nozzle of the electronic atomization inhaler including a vent pipe and an opening portion of a liquid container is necked;

FIG. 6 illustrates a cross-sectional view of another electronic cigarette;

FIG. 7 illustrates a perspective view of a sleeve that is positioned around an electronic cigarette; and

FIG. 8 illustrates a perspective view of a cigarette container.

### DETAILED DESCRIPTION OF THE PREFERRED EMBODIMENTS

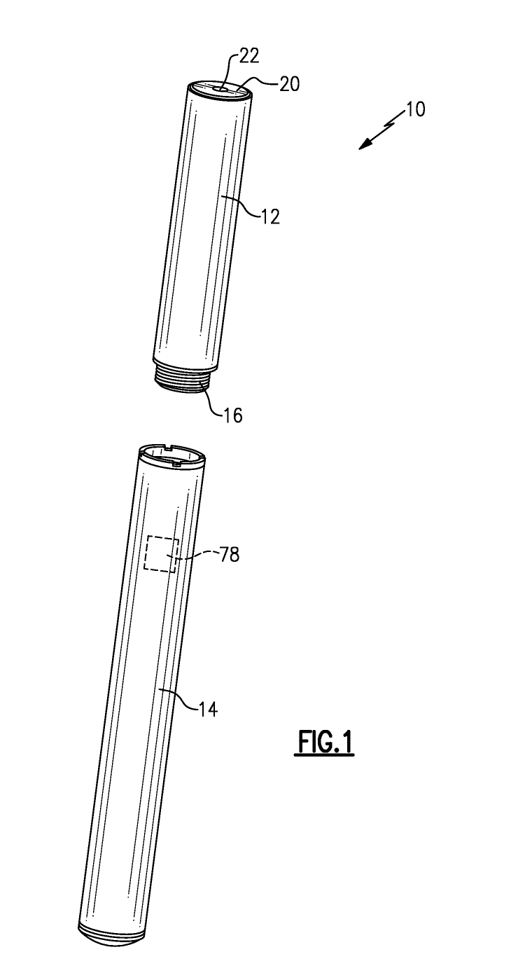

FIG. 1 shows a first example electronic atomization inhaler 10. In one example, the electronic atomization inhaler 10 is an electronic cigarette, an electronic cigar, an electronic cigarillo, or an electronic inhaler. The electronic atomization inhaler 10 atomizes liquid to a vapor, which can be inhaler by a user.

The electronic atomization inhaler 10 includes an atomization nozzle 12 and a power source 14 that are electrically and mechanically connected to each other by a threaded electrode 16. The power source 14 provides electric energy to the atomization nozzle 12. The atomization nozzle 12 can be disposable. That is, the atomization nozzle 12 can be replaced after the liquid 62 in the atomization nozzle 12 is depleted.

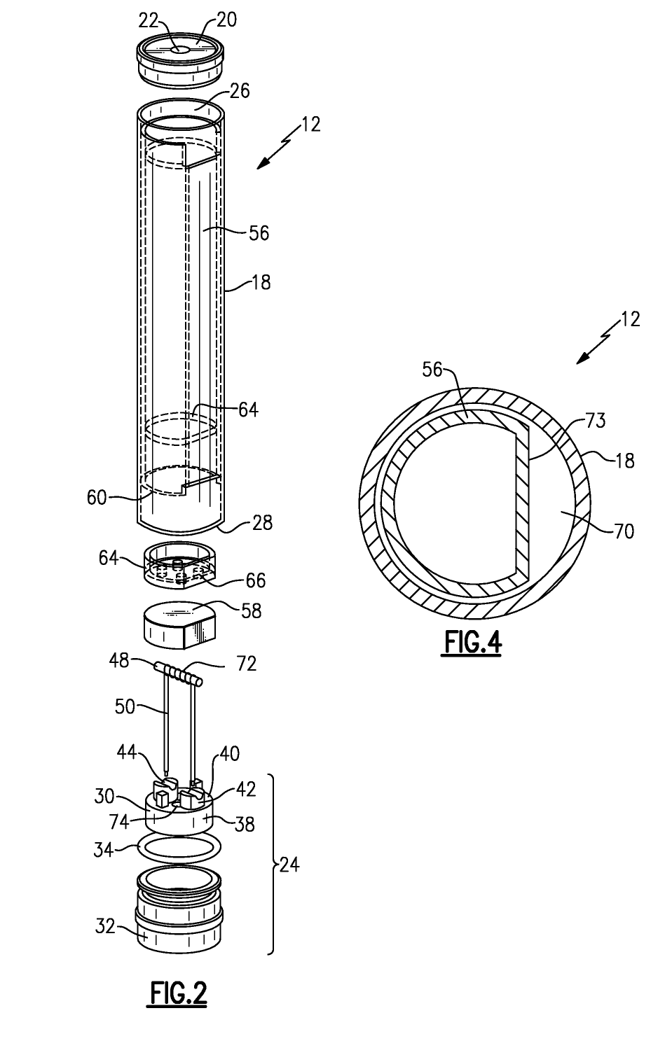

As shown in FIGS. 2, 3 and 4, the atomization nozzle 12 of the electronic atomization inhaler 10 includes a cylindrical body 18. A cap 20 including an air outlet 22 is inserted into an opening 26 at a front end of the cylindrical body 18 (the front end is the end inserted into the user's mouth). When a user inhales the atomized vapor from the electronic atomization inhaler 10, the atomized vapor flows through the air outlet 22 and is inhaled.

A stopper 24 is partially received in an opening 28 at a rear end of the cylindrical body 18. The stopper 24 includes a heater retainer 30 and a hollow cylinder sealing base 32. An outer surface of the sealing base 32 is secured in the opening 28 by a sealing ring 34, which also prevents the leakage of liquid 62, as explained below. The heater retainer 30 is elastic and made of a temperature resistant material, such as silica gel, ceramics, silicon rubber, and a seal is created by the stopper 24 and an inner wall of the sealing base 32.

The heater retainer 30 defines an air inlet 36. The heater retainer 30 includes a body 38 having an upper surface 40 and two columns 42 that project from the upper surface 40. The two columns 42 have upper surfaces that include substantially semicircular grooves 44. The heater retainer 30 also includes openings 46 that extend through the two columns 42 and the body 38 of the heater retainer 30.

A heating element 48 is received in the substantially semicircular grooves 40 of the two columns 42. Two electrical leads 50 extend from the heating element 48 and pass through the openings 46 in the heater retainer 30. The two electrical leads 50 are in electrical connection with the power source 14 by the threaded electrode 16. The end of the sealing base 32 includes connecting threads that form one electrode of the threaded electrode 16 (the other electrode of the threaded electrode 16 is not shown).

The electronic atomization inhaler 10 includes a pressure sensor (not shown) that detects when a user employer the electronic atomization inhaler 10 to determine suction and if the user is inhaling. When the pressure sensor detects a pressure change caused by suction, the power source 14 supplies power to the heating element 48.

In one example, the temperature of the heating element 48 is approximately 200° F. to approximately 400° F. The temperature selected for the heating element 48 depends upon the additive employed in a liquid container 56 (discussed below). The size of the heating element 48, for example, the length and the diameter of the heating element 48, can also determine the temperature of the heating element 48. The size of the heating element 48 can be selected for each specific additive to heat the heating element 48 to the proper temperature for the additive.

The sealing base 32 defines a central opening 52 in fluid communication with the air inlet 36. A surface of an inner cavity of the sealing base 32 includes an annular projection 54. The central opening 52 is located centrally within the annular projection 54. A lower surface of the heater retainer 30 contacts an upper surface of the annular projection 54 of the sealing base 32 to retain the heater retainer 30 in position.

A liquid container 56 including an opening 60 is located within the cylindrical body 18 and retained therein by the cap 20 and the stopper 24. The liquid container 56 is filled with the liquid 62 that is atomized as a vapor to be inhaled.

A liquid guide device 58 absorbs the liquid 62 stored in the liquid container 56, preventing the liquid 62 from flowing out of the opening 60. The liquid guide device 58 can be made of fiber, cotton, sponge or any temperature-resistant, nontoxic and porous material. The liquid guide device 58 is located between the stopper 24 and the liquid container 56. When the heater retainer 30 contacts a lower surface of a liquid guide device 58, the heating element 48 can contact a lower surface of the liquid guide device 58.

An interior surface of the liquid container 56 includes an annular projection 64. A gasket 66 is inserted through the opening 28 and is received in an interior of the liquid container 56 and is disposed next to the annular projection 64. The gasket 66 includes liquid guide holes 68 that can slowly guide the liquid 62 in the liquid container 56 to the liquid guide device 58.

The liquid guide device 58 and the gasket 66 are inserted within the liquid container 56 through the opening 28 such that the gasket 66 is located between the liquid guide device 58 and the annular projection 64 of the liquid container 56. The gasket 66 and the annular projection 64 can prevent the liquid guide device 58 from over inserting into the liquid container 56 during the assembling of the liquid guide device 58 or when the rear end stopper 24 presses against the liquid guide device 58.

An air passage 70 is located between the cylindrical body 18 and the liquid container 56 and is in communication with the air outlet 22 and the air inlet 36. The air passage 70 can be uniformly distributed around a periphery of the liquid container 56 or can be distributed on one or several sides of the liquid container 56. In one example, one side wall of the liquid container 56 includes a flattened portion 73 to define the air passage 70 between the liquid container 56 and the cylindrical body 18 (FIG. 3).

A communication passage 74 (atomization chamber) is located between the columns 42 of the heater retainer 30 and provides for air circulation. The communication passage 74 is provided between the rear end stopper 24 and the liquid guide device 58. The heater retainer 30 is in direct contact with the heating element 48, allowing the heater retainer 30 to absorb some of the heat generated by the heating element 48 so that the temperature of air in the communication passage 74 and the liquid 62 in the liquid guide device 58 above the communication passage 74 can be maintained at a relatively high degree, even when the heating element 48 stops heating.

The heating element 48 heats and atomizes the liquid 62 in the liquid container 56. The heating element 48 extends between the columns 42 of the heater retainer 30 and is located within the grooves 44, allowing the heating element 48 to contact the liquid guide device 58. The heating element 48 absorbs the liquid 62 on the liquid guide device 58 such that liquid 62 droplets diffused in the communication passage 74 and on the heating element 48 may be atomized by the heating element 48 and then directed to the air passage 70 and the air outlet 22 for inhalation. A temperature resistant fiber core 72 can also be wound around the heating element 48 so that the liquid 62 droplets diffused from the liquid guide device 58 can be further absorbed for heating and atomization by the heating element 48.

In another embodiment (not shown), a center of an upper surface of the heater retainer 30 includes a concave chamber, and the air inlet 36 communicates with the concave chamber. The heating element 48 radially bridges a top of a side wall of the concave chamber. The peripheral side wall of the concave chamber includes a through hole. The side wall of the top end portion of the heater retainer 30 keeps the heating element 48 below the liquid guide device 58, while the concave chamber forms the communication passage 74. The atomized vapor passes through the through hole on the side wall of the concave chamber to enter the air passage 70.

In another example (not shown), a lower surface of the liquid guide device 58 is provided with a diffusion cushion. The heating element 48 contacts the lower surface of the diffusion cushion to more evenly diffuse the liquid 62 on the liquid guide device 58. In one example, the diffusion cushion is made of flaky material. The diffusion cushion can be bonded on the lower surface of the liquid guide device 58.

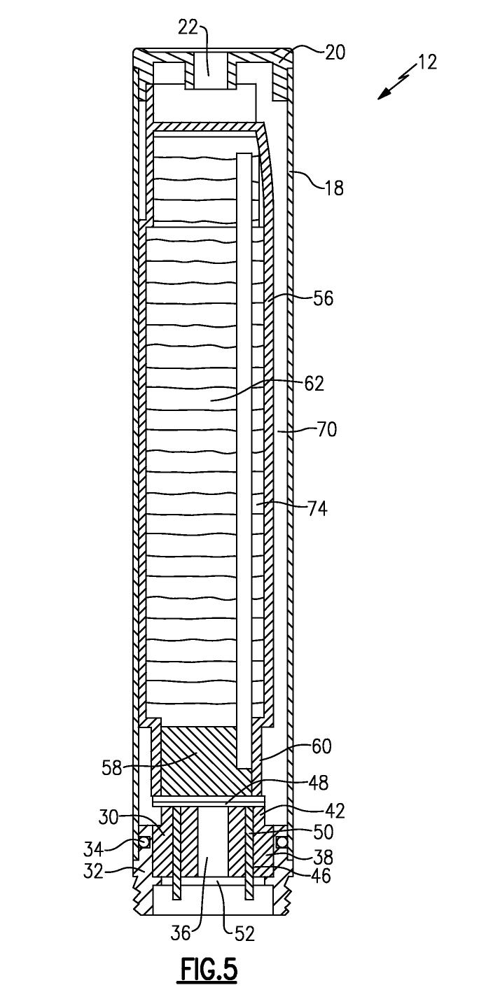

In one example shown in FIG. 5, one end of a vent pipe 76 is inserted into the liquid guide device 58 without penetrating entirely through the liquid guide device 58, and the other end of the vent pipe 76 extends into the liquid container 56. The liquid 62 in the liquid container 56 can permeate more easily into the liquid guide device 58. Air outside the liquid container 56 may penetrate into the liquid container 56 slowly along the vent pipe 76 to prevent negative pressure in the liquid container 56. The opening 60 of the liquid container 56 can define a neck having a smaller inner diameter than a remainder of the liquid container 56. The entire liquid guide device 58 is inserted into the opening portion 28 of the liquid container 56.

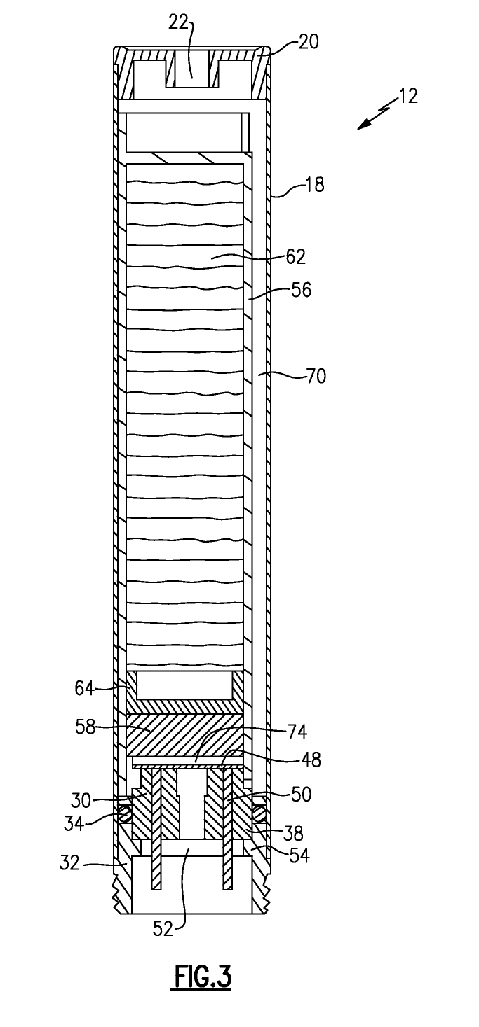

Returning to FIGS. 2 and 4, the liquid container 56 contains a liquid 62 including at least one additive. In one example, the liquid container 56 contains liquid nicotine, liquid eggplant, or liquid eggplant extract. In another example, the liquid container 56 contains an additive, as summarized in the chart below. In another example, the liquid container 56 contains one of liquid nicotine, liquid eggplant, liquid eggplant extract plus one or more additives.

Some of the possible additives are listed below:

| ADDITIVE | BENEFITS |
|---|---|
| Sildenafil | Treats erectile dysfunction |
| Natural herbs | |
| Ashwagnanda | Ayurvedic, increase sexual energy, |
| | promotes production of semen |
| Ginko Biloba | Positive effects on sexual response cycle |
| Epimedium | Increases sperm and blood testosterone |
| L-Arginine | Reduces impotence and increases |
| | testosterone |
| St. John's Wort | Reduces anxiety |
| Yohimbe | Aphrodisiac |
| Kava | Reduces anxiety and stress |
| Maca root | Increases sexual drive and increases sexual |
| | function |
| Muira puama | Increase libido and youthful feelings |
| Saw palmetto | Optimizes sexual function |
| Ginseng | Increases blood flow to sexual organs |
| Vitamins (B12, etc.) | Reduce vitamin deficiency and increase |
| | energy |
| Valerian root | Relaxation and treatment of sleep |
| | disorders |
| Royal Jelly | Improves immune system |
| Chinese red ginseng/Siberian | Stimulates sexual function and increases |
| ginseng | energy |
| He shou wu/fo ti | Builds energy |
| Bitter orange | Weight loss |
| Coenzyme Q110 | Converts food into energy |
| DHEA | Increases DHEA hormone levels |
| Catuaba | Increases libido and sexual stimulation |
| 5-HTP | Restore serotonin levels |
| Lemon Balm | Acts as a sedative and an anti-bacterial |
| Lavender | Reduces anxiety and stress |
| Passion flower | Reduces anxiety |
| Chamomile | Reduces anxiety |
| Acetyle-L-Carnitine | Increases energy |
| DHA | Increases brain function |
| Choline | Increases memory |
| Blue green algae extract | Increases mental alertness and memory |
| Rhodiola rosea | Promotes healthy aging and reduces |
| | oxidative stress |
| DMAE | Treats attention deficit hyperactivity |
| | disorder |
| Eleuthero root | Restores memory |
| Cinnamon | Restores metal alertness |
| Turmeric | Provides anti-inflammatory features |
| Rosemary | Protects the brain |
| Omega 3 | Fights against mental disorders and |
| | depression |
| Vitamin C | Reduces learning defects |
| Vitamin B | Regulates mood and prevents metal |
| | fatigue |
| Serotonin | Provides relaxation |
| Kola nut | Reduces fatigue |
| Human Growth Hormone | Diet Suppressant |
| Green Tea Extract | |
| Testosterone | |
| Caffeine | |
| Viagra | |
| Insulin | |
| Cialis | |
| Oxytocin | Increases orgasm and euphoria |
| Estrogen | |
| Antibiotics | |
| Antacid | |
| Menthol | |
| Natural nicotine | |
| Viagra and Oxytocin | |
| Cialis and Oxytocin | |
| Subutex | |
| Niacin | |
| Medical marijuana | |

If the liquid container 56 of the electronic atomization inhaler 10 includes nicotine, the amount of nicotine can vary depending upon the amount of nicotine desired by the person. For example, if the person is trying to quit smoking, the person may want to, over time, purchase an electronic atomization inhaler 10 including a reduced amount of nicotine until the person eliminates nicotine in the electronic atomization inhaler 10 completely.

Various additives can be added to the liquid container 56 and inhaled. Any additive or combination of additives can be added to the electronic atomization inhaler 10, both with or without liquid nicotine, liquid eggplant extract, or liquid eggplant. In one example, the additives are flavoring, drugs, pharmaceuticals, a vitamin, an herb, a homeopathic additive, or a dietary supplement. Employing pharmaceuticals in an electronic atomization inhaler 10 provides the benefit of allowing a medicine to be inhaled instead swallowed in a pill form. This can be beneficial for a person who has difficulty swallowing pills.

The power source 14 of the electronic atomization inhaler 10 also includes a microchip 78 (show schematically) that controls various parameters of the electronic atomization inhaler 10. The microchip 78 can be installed at the manufacturer.

In one example, the microchip 78 controls the temperature of the heating element 48. In another example, the microchip 78 controls the duration of each puff on the electronic atomization inhaler 10. In another example, the microchip 78 controls the length of time between puffs of the electronic atomization inhaler 10.

In one example, the atomization nozzle 12 including the additive is replaceable. In this example, the same power source 14 can be employed. As the length and diameter of the heating element 48 determines the heating temperature, the desired heating temperature can be attainable with different atomization nozzles 12 as the temperature can be determined by the size heating element 48 in the atomization nozzle 12.

FIG. 6 illustrates a second example electronic cigarette 110. The electronic cigarette 110 includes a suction portion 112 including a passage 114 through which a person inhales air and other materials within the electronic cigarette 110, as explained below. The suction portion 112 is attached to a cartridge 116. In one example, the cartridge 116 includes a heating element 118. In one example, the heating element 118 is tubular. In one example, a piece of material 120 including nicotine surrounds the heating element 118. In another example, the piece of material 120 includes eggplant, eggplant extract or an eggplant additive substitute, which simulates the effects of nicotine. In another example, the electronic cigarette 110 does not include the piece of material 120.

Outside and surrounding the heating element 118 or the piece of material 120 is a piece of material 122 including a liquid additive, as discussed and listed above. In one example, the liquid is oil. However, the additive added to the electronic cigarette 110 can be in any form. For example, the additive can be a liquid, an oil, in crystal form, a solid capsule, a solid tablet, a powder, a vapor, or in its natural form (for example, raw herbs). In one example, the material 122 is fabric. In one example, the material 122 is a sponge. In one example, the additive is a homeopathic additive. In one example, the additive is a vitamin. In one example, the additive is a pharmaceutical or drug.

The heating element 118, the piece of material 120 (optional) and the piece of material 122 including the liquid additive are secured together to define the cartridge 116. In one example, the components are secured by a piece of tape 124. The heating element 118 also functions as an atomizer that produces a mist.

The electronic cigarette 110 includes a power source 126 that activates the heating element 118 in response to a signal. In one example, the power source 126 is a battery. The electronic cigarette 110 also includes a light cover 128 that glows when activated by the signal. In one example, the light cover 128 is an LED light cover. The light cover 128 can produce any color light. The electronic components of the electronic cigarette 110 can be connected by a plurality of wires. A cover 136 encases the components of the electronic cigarette 110 to simulate the appearance of an actual cigarette. In one example, the cover 136 is made of plastic. An outer cover 146 can be located outside the cover 136 to create the appearance of an actual cigarette. In one example, the cover 146 is made of paper and wrapped around the cover 136.

When a user places the suction portion 112 of the electronic cigarette 110 into their mouth and inhales, a pressure sensor 132 detects the inhalation. As the person inhales, air flows through passages 134 in the light cover 128, through the heating element 118, and through the passage 114 of the suction portion 112. The pressure sensor 132 then sends a signal to the power source 126 to activate the heating element 118 to heat the piece of material 120 (optional) and the piece of material 122 including the additive, vaporizing the nicotine and the additive so that it can be inhaled into the person's lungs in a vapor form that is later exhaled as a vapor and has the appearance of smoke. The pressure sensor 132 also sends a signal to activate the light cover 128 to glow to simulate the lighted end of a cigarette.

In another example the electronic cigarette 110 is rechargeable. In one example, a liquid additive is contained inside a capsule. When the capsule is installed in the electronic cigarette 110, the capsule is punctured, allowing the liquid additive to be inhaled during use of the electronic cigarette 110. In another example, the additive added to the electronic cigarette 110 can be in any form. For example, the additive can be a liquid, an oil, in crystal form, a solid capsule, a solid tablet, a powder, a vapor, or in its natural form (for example, raw herbs). When inhaled, the additive is vaporized into a vapor.

If the electronic cigarette 110 includes nicotine, the amount of nicotine in the material 120 can vary depending upon the amount of nicotine desired by the person. For example, if the person is trying to quit smoking, the person may want to, over time, purchase electronic cigarettes 110 including a reduced amount of nicotine until the person eliminates nicotine in the electronic cigarettes 110 completely.

Various additives can be added to the material 122 and inhaled when the person applies suction to the suction end 112 of the electronic cigarette 110. The table summarizing various additives and their benefits is listed above.

Any additive or combination of additives can be added to the electronic cigarette 110, both with or without nicotine or eggplant extract. In one example the additive is part of the cartridge 116. However, the additive can be located in other areas of the electronic cigarette 110.

In another example, the additives are flavoring, drugs or pharmaceuticals. Employing pharmaceuticals in an electronic cigarette 110 provides the benefit of allowing a medicine to be inhaled instead swallowed in a pill form. This can be beneficial for a person who has difficulty swallowing pills.

The additives can provide many benefits of effects. For example, as stated above, the additives can suppress appetite, increase energy, act as a sleep aid, increase sexual desire/enhancement, reduce anxiety, reduce hangovers, increase mental and neurological function, increase memory, increase physical endurance, provide daily vitamins, assist in waking up, increase alertness, reduce bad breath (for example, act as a breath mint) and provide an anti-aging function.

The additives can also reduce the risk of cancer, provide pain relief, reduce arthritis, act as a diet suppressant, reduce weight gain, reduce Migraine and headaches, act as an anti-depressant, replace hormones, reduce inflammation, prevent insomnia, reduce allergies, reduce acid indigestion, assist in digestion, increase fertility, act as a diuretic, reduce addition, act as a probiotic, increase concentration, prevent ADD or ADHD, reduce the effects of menopause, reduce the effects of premenstrual syndrome, reduce sea sickness, reduce motion sickness/dizziness, increase prostrate health, reduce gas, maintain thyroid health or be hormones.

In another example, a portion of the electronic atomization inhaler 10 or the electronic cigarette 110 can be colored to indicate the additive or the function/benefits provided by the additive. In this example, there is a known correlation between the color employed and the additive.

FIG. 7 illustrates a sleeve 210 located outside the electronic atomization inhaler 10 or the electronic cigarette 110 to provide a decorative or designer appearance. The sleeve 210 can be decorative and provides a custom appearance. In one example, the sleeve 210 is made of a flexible material. In one example, the sleeve 210 is made of spandex or rubber. This allows the sleeve 210 to be pulled over the electronic cigarette 110. When the sleeve 210 is to be removed from the electronic cigarette 110, the sleeve 210 can be pulled away from and removed from the electronic cigarette 110. As the sleeve 210 is flexible, the sleeve 210 can be easily added or removed. The sleeve 210 can have any color, any pattern or any texture. In one example, the sleeve 210 has an animal print pattern, such as a leopard, cheetah, tiger, or snake.

The sleeve 210 can also be customized or selected based on the preferences of the person using the electronic cigarette 110. For example, the sleeve 210 can be decorative to make the electronic cigarette 110 more fashionable. In another example, the sleeve 210 can be functional. In this example, the sleeve 210 can include a colored portion that informs the person of the additive(s). That is, there is a known or pre-determined correlation between the color of the sleeve 210 and the additive contained in the liquid container 56.

In another example, the sleeve 210 is attached to the electronic atomization inhaler 10 or the electronic cigarette 110 prior to purchase. In this example, the person cannot change the sleeve 210, and the person would select the electronic atomization inhaler 10 or the electronic cigarette 110 with the sleeve 210. In one example, the sleeve 210 is made of a paper or fabric material that is glued to the electronic cigarette 110. The sleeve 210 can have any color, pattern or texture. Additionally, other features can be added to the cover sleeve 210 to provide additional designs, such as lace, sequins, glitter, etc. The sleeve 210 can be made of leather, a leather like material, or have a leather appearance.

In another example, the sleeve 210 is transparent, which allows the components of the electronic cigarette 110 to be viewed.

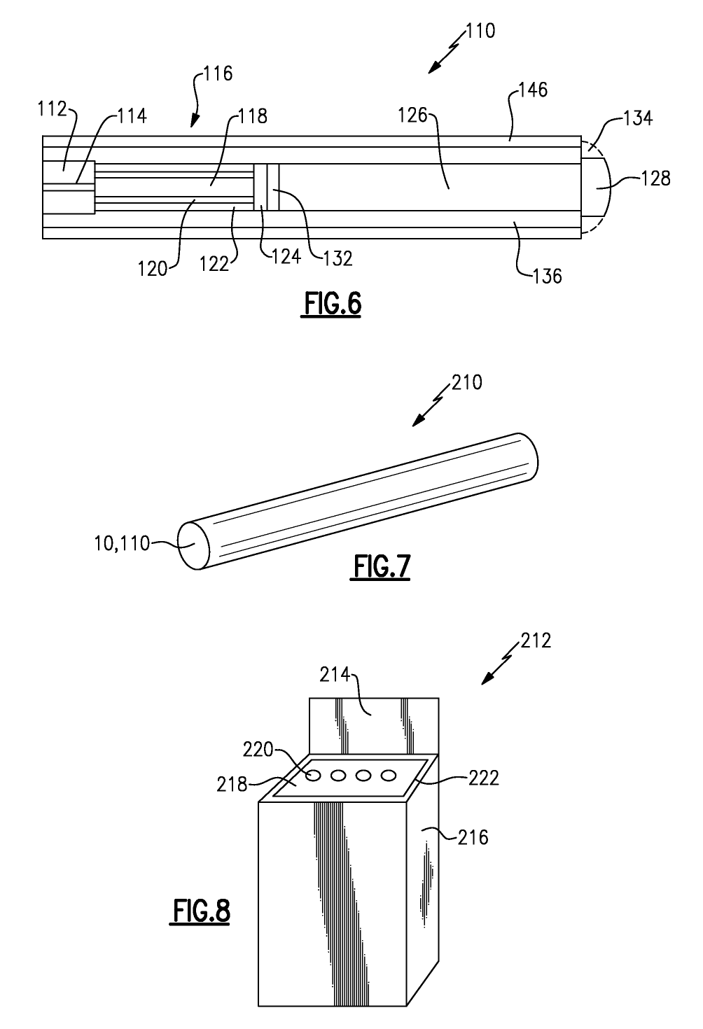

FIG. 8 illustrates a decorative cigarette case 212 that holds a cigarette package 222. The removable cigarette case 212 includes a body portion 216 and a lid 214 that closes to cover over the electronic atomization inhaler 10 or the electronic cigarette 110 received in the decorative cigarette case 212. The decorative cigarette case 212 includes a solid portion 218 that includes at least one hole 220 that receives an atomization inhaler 10 or an electronic cigarette 110. The decorative cigarette case 212 can be customized and decorated in any manner, including any color, fabric, material, etc.

The foregoing description is only exemplary of the principles of the invention. Many modifications and variations are possible in light of the above teachings. It is, therefore, to be understood that within the scope of the appended claims, the invention may be practiced otherwise than using the example embodiments which have been specifically described. For that reason the following claims should be studied to determine the true scope and content of this invention.

What is claimed is:

1. An electronic inhaler comprising:
a cylindrical body;
a liquid container inside the cylindrical body that contains a liquid including an additive;
a heating element to heat the liquid including the additive to a vapor;
an air passage located between the cylindrical body and the liquid container in communication with an air outlet to allow the vapor to flow through the air outlet.

2. The electronic inhaler as recited in claim 1 wherein the cylindrical body, the liquid container, the heater and the air passage define an atomization nozzle.

3. The electronic inhaler as recited in claim 2 including a power source electrically and mechanically connected to the atomization nozzle.

4. The electronic inhaler as recited in claim 1 including a cap having the air outlet inserted into a front end of the cylindrical body and a stopper having an air inlet inserted into a rear end of the cylindrical body.

5. The electronic inhaler as recited in claim 4 wherein the stopper includes a heater retainer that retains the heating element and a sealing base that seals the stopper relative to the cylindrical body.

6. The electronic inhaler as recited in claim 4 wherein the heater retainer includes a base and two columns each including a groove that extend from the base, and the heating element is received in the groove of each of the two columns.

7. The electronic inhaler as recited in claim 6 wherein the body and the two columns each include an opening, and an electrical lead is located in each of the openings and is in electrical connection with a power source.

8. The electronic inhaler as recited in claim 1 including a gasket located in the cylindrical body near an opening of the liquid container that controls a flow of the liquid from the liquid container and a liquid guide device that absorbs the liquid that that flows through the liquid container.

9. The electronic inhaler as recited in claim 1 wherein the liquid further includes nicotine.

10. The electronic inhaler as recited in claim 1 wherein the liquid further includes an eggplant extract.

11. The electronic inhaler as recited in claim 1 wherein the additive is one of a flavor, a pharmaceutical, an herb, a vitamin, a dietary supplement or homeopathic.

12. The electronic inhaler as recited in claim 11 wherein the liquid further includes nicotine.

13. The electronic inhaler as recited in claim 1 wherein a decorative sleeve is located on an exterior of the electronic inhaler.

14. The electronic inhaler as recited in claim 1 wherein the decorative sleeve is made of a flexible material.

15. An electronic inhaler comprising:
an atomization nozzle including a cylindrical body, a liquid container inside the cylindrical body that contains a liquid including an additive, a heating element to heat the liquid including the additive to a vapor, and an air passage located between the cylindrical body and the liquid container in communication with an air outlet to allow the vapor to flow through the air outlet, wherein the additive is one of a pharmaceutical, a vitamin, a flavor, a dietary supplement, and an herb; and
a power source electrically and mechanically connected to the atomization nozzle.

16. The electronic inhaler as recited in claim 15 including a cap having an air outlet inserted into a front end of the cylindrical body and a stopper having an air inlet inserted into a rear end of the cylindrical body, wherein the stopper includes a heater retainer that retains the heating element and a sealing base that seals the stopper relative to the cylindrical body.

17. The electronic inhaler as recited in claim 16 wherein the heater retainer includes a base and two columns each including a groove that extend from the base, the heating element is received in the groove of each of the two columns, and the body and the two columns each include an opening, and an electrical lead is located in each of the openings and is in electrical connection with the power source.

18. The electronic inhaler as recited in claim 15 including a gasket located in the cylindrical body near an opening of the liquid container that controls a flow of the liquid through the liquid container and a liquid guide device that absorbs the liquid that that flows through the liquid container.

19. The electronic inhaler as recited in claim 14 wherein the liquid further includes nicotine.

20. The electronic inhaler as recited in claim 14 wherein the liquid further includes eggplant extract.
